(19) United States
(12) Patent Application Publication
(10) Pub. No.: US 2010/0040654 A1
(43) Pub. Date: Feb. 18, 2010

(54) EXFOLIATING AND MOISTURIZING COMPOSITION AND METHOD

(76) Inventors: Masoomeh Wake, Lebanon, NJ (US); John Woodruff, Dorset (GB); Janet Amitrano, Milford, PA (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/582,483

(22) Filed: Oct. 20, 2009

Related U.S. Application Data
(63) Continuation-in-part of application No. 11/377,982, filed on Mar. 17, 2006.
(60) Provisional application No. 60/663,073, filed on Mar. 21, 2005.

(51) Int. Cl. A61K 8/02

(52) U.S. Cl. 424/401

(57) ABSTRACT

An exfoliating and moisturizing composition for the skin includes a wax component having a melting point of at least 35° C. and an exfoliant mineral component dispersed in the wax component. The mineral component has a particle size of no more than 3 microns. Minerals may include a clay and/or a mineral salt. The composition may include water and surfactants. Also disclosed is a method for exfoliating the skin through the use of the composition.

### CROSS-REFERENCE TO RELATED APPLICATIONS

This application is a continuation-in-part of U.S. patent application Ser. No. 11/377,982 filed Mar. 17, 2006, which claims priority of U.S. Provisional Patent Application No. 60/663,073 filed Mar. 21, 2005, entitled “Exfoliating and Moisturizing Composition and Method.”

### FIELD OF THE INVENTION

The present invention includes a personal care composition, for use on the body, feet, and/or face, comprising a clay, minerals, one or more waxes, emulsifier and water. The composition comprises one or more skin exfoliating ingredients contained in a total amount of at least 60.00% by weight of the composition, the composition being capable of exfoliating, polishing, gentle cleansing, and moisturizing the skin, with no irritation. The composition can be in the form of a stick, tablets, or other solid or semisolid forms. An embodiment produces particles whose size and appearance provides the user with a visual signal about the degree of exfoliation occurring. The composition has a “self-regulating” property such that the skin is exfoliated to the extent needed by the skin itself.

### BACKGROUND OF THE INVENTION

Many substances are applied directly to the skin of humans for purposes such as to alter the subject's appearance, to protect the subject from the environment, or to produce a biological change in the subject's skin or other tissues for therapeutic, preventative or cosmetic purposes. Because these substances are applied directly to the surface of the skin, they are generally referred to as “topical” products; such topical products can include topically applied substances such as, but not limited to, cosmetics, over-the-counter products, prescription drugs, and a variety of other products such as soaps, detergents and the like.

Topical products are available in a variety of forms, including solids, liquids, suspensions, semisolids (such as creams, gels, ointments, pastes or sticks), powders or finely dispersed liquids such as sprays or mists. Examples of topical products commonly classified as “cosmetics” include skin care products such as creams, lotions, moisturizers, and “treatment cosmetics” such as exfoliants and/or skin cell renewal agents. Other examples of topical products include hand, facial and body soaps and detergents, and other forms of skin cleansers, as well as household detergents.

Exfoliants are one class of topical preparation. They are used to enhance the normal process of desquamation, and thereby soften and renew the surface of the skin. As will be explained below, the present invention is directed to exfoliating compositions and methods which are highly effective yet nonirritating, even with regard to sensitive skin.

In many instances, topical products contain chemicals, which may produce “irritation” including various inflammation symptoms or signs, when applied to the skin or mucosa (both of which will be referred to as “skin” herein). The occurrence, frequency and nature of topical product induced irritation varies from user to user. Typical symptoms of irritation include itching (pruritus), stinging, burning, tingling, “tightness,” erythema (redness) or edema (swelling). The irritation response may be due to a direct effect on the skin of certain topical product chemicals or to a response by the individual's immune system directed toward one or more specific chemicals alone, or in combination with a response to one or more skin components The sensory nerves of the skin can be considered to be a “final common pathway” for the many irritating conditions which may be ultimately sensed as itch, including chemical exposure and/or environmental exposure (such as that which produces dry, itchy skin). No matter what the ultimate cause of itch, the sensation experienced is the same and provokes the desire to scratch. While a certain amount of scratching generally may immediately relieve an itch, extensive scratching at a particular site may often lead to further irritation, skin damage and possibly infection.

Many ingredients used in topical products, and in particular in exfoliants, are known irritants, or are potentially irritating, especially to people with “sensitive skin.” Sensitive skin is generally considered to be a skin type that is more sensitive than others to one or more external factors, such that this type of skin is easily irritated. These factors can be ingredients which are found, for example, in fragrances, preservatives, solvents, and other ingredients that might otherwise be considered inert components of the products. Additionally, many of the ingredients used as active ingredients in topical products, including chemicals that may also be classified as drugs, may produce irritation when applied to the skin. These include, but are not limited to such ingredients as exfoliants, skin cell renewal agents, anti-inflammatory agents, skin protective agents and many others. Where more than one chemical irritant is present in a composition, their irritating effects may be additive. Furthermore, there are some chemical ingredients, which while not irritants by themselves, may react with one another to, or when applied to the environment of the skin, form one or more new chemicals, which are irritating.

Many chemicals directly trigger skin irritation. Some chemicals which would not normally cause irritation indirectly cause the skin to become more sensitive to other chemicals or environmental conditions. Some chemicals which act as skin “exfoliants” are retinoids, retinoic acid and retinol; carboxylic acids including alpha-hydroxy acids (e.g. lactic acid, glycolic acid), beta-hydroxy acids (e.g. salicylic acid, beta-hydroxy butyric acid); alpha-keto acids, acetic acid, oxalic acid, and malic acid among others. Such compounds may be direct irritants, and they also may cause the skin to become more sensitive to irritation triggered by other topically-applied chemicals such as moisturizers, sunscreens, surfactants (e.g. soaps) and other topical products. Exfoliants and other ingredients may also increase the skin's sensitivity to environmental conditions, such as wind, cold temperature, dry air or sunlight.

Conversely, environmental factors may increase the skin's sensitivity to chemicals in topical products by reducing the epidermal skin's “barrier function.” The barrier function refers to the property of skin which minimizes absorption or passage of potentially harmful chemicals or substances through the outer “dead” cell layer of the epidermis. A common environmental factor, such as exposure to low humidity or prolonged exposure to refrigerated air, can result in itchy skin. Agents such as soaps, detergents, cleansing products, shaving creams, and other products which remove some of the skin's protective lipids and/or secretions may also increase the skin's permeability and sensitivity to topically applied chemicals, which alone would not otherwise have produced irritation.

Whatever the exact cause of irritation, attempts have been made to reduce the irritation potential of topical products by identifying those chemicals which tend to cause irritation, and either reducing their concentration or eliminating them from the product(s). Many of these products are advertised to consumers as “hypoallergenic” to designate the product's reduced tendency to cause irritation on individuals with sensitive skin. However, it is often not feasible or practical to identify or eliminate all of the irritating chemical(s), particularly when the irritating chemical(s) is(are) the active ingredient of the product or required for formulation, preservative or other functional reasons.

Exfoliating agents affect the skin by either chemical or mechanical action, or both. Mechanical exfoliants physically remove cells from the surface of the skin. Mechanical exfoliants not only aid in sloughing unwanted cells, they refine the texture of the skin and stimulate cell renewal. They further benefit skin by leaving it ideally prepared for subsequent caring or intensive treatments. The two most common types of mechanical exfoliants are scrubs and peels. These cleansing compositions contain abrasives, and are generally characterized with the disadvantage of having an unpleasant abrasive and/or sandy feel, and which compositions may cause irritation with prolonged scrubbing.

Chemical exfoliants exert a chemical effect on the skin. Certain agents, referred to as sloughing accelerators, are various combinations of acids and other exfoliation ingredients, which are believed to act by dissolving the intercellular cement which holds the dead cells on the surface together. Once the intercellular cement is dissolved, cells are no longer attached and are easily removed. The two most prominent sloughing, accelerators are alpha-hydroxy acids (e.g. lactic acid, glycolic acid) and beta-hydroxy acids (e.g. salicylic acid). In some exfoliating cleansers, these hydroxy acids are combined with plant extracts that add skin soothing and moisturizing properties. A problem is that hydroxy acids, when used at concentrations high enough to exfoliate, are known to cause skin irritation and rashes, and this danger of irritation is even higher for persons that have sensitive skin.

There are many exfoliating products in the personal care market. However, the majority are very irritating to the skin because they are either soap or surfactant based (e.g. sodium methyl cocoyl taurate, sodium lauryl sulfate, cocamidopropyl betaine); have coarse particulates (e.g. sugars, salts, beads, seeds); have a high level of harsh or marginally effective exfoliants (e.g. alpha-hydroxy acids, beta-hydroxy acids, salicylic acid, or botanicals); low levels of moisturizers, or combinations thereof.

The present invention relates to a method of exfoliating the skin by combining wax and exfoliating clay and minerals together to enhance the normal process of desquamation of the stratum corneum. The normal daily sloughing of dead skin from an average individual (process of desquamation) has been estimated to cause a loss of up to 14 grams of tissue per day. This loss of outer skin cells is constantly replenished with cells from lower layers of the epidermis. Thus, the layers of the epidermis are composed of cells moving up towards the surface in successive stages of differentiation. The outer layer of skin cells are dead, and they are finally sloughed off and lost to the environment. When desquamation does not take place regularly, the surface of the skin tends to become rougher and more wrinkles and other undesirable effects appear on the surface of the skin.

Exfoliation is often used to rejuvenate and enhance the health of the skin, and may be used in addition to, or as an alternative to the natural desquamation process. The present invention combines low and high molecular weight waxes. The high molecular weight waxes are believed to attach themselves to the dead cells while the lower molecular weight waxes provide moisturization. The fine minerals provide the mechanical abrasion and gently cleansing process. The fine particle size of the minerals (0.60-3 microns) does not damage the skin as some commercially available exfoliants do (e.g. apricot seeds). The present invention, when embodied as an exfoliating stick, tablet or other product form, leaves the user's exfoliated skin to appear fresh and healthy as it removes the dull layer of dead skin, accompanied with non-soap cleansing, leading to less clogged pores while at the same time moisturizes the skin to minimize irritation and dryness.

There is a long felt, substantial, practical and commercial need in the field of exfoliants and related skin care products for a composition and/or method that will reduce or prevent irritation caused by such products.

The skin types among individuals within a population vary, and the production of a variety of exfoliants is desirable in order to meet these various individual skin care needs. Thus, there is a continued need to find additional alternative ways of aiding the sloughing (desquamation) ability of the skin and promoting its health for various skin types.

Embodiments of the present invention address these problems, as they include a combination of a wax and exfoliating particles which both enhances the exfoliating process (desquamation) and provides the user with gentle cleansing and simultaneous moisturizing with no irritation.

### BRIEF DESCRIPTION OF THE INVENTION

Disclosed herein is an exfoliating and moisturizing composition for the skin. The composition includes a wax component having a melting point of at least 35° C. and an exfoliant mineral component dispersed in the wax component. The mineral component has a particle size of no more than 3 microns. In particular embodiments, the mineral component includes a clay, and the clay may be an anionic clay. Some examples of clay which may be utilized in the invention include bentonite, kaolin, hectorite, montmorillonite, and various combinations of the foregoing. The mineral component may include a salt of a metal such as Al, Mg, Ca, and Zn. The salt may be an inorganic salt such as a silicate or carbonate, or it may be a fatty acid salt such as a stearate. In particular instances, the mineral component has a particle size in the range of 0.6-3.0 microns.

In particular embodiments, the mineral component comprises 3-80% by weight of the composition, and in specific instances, 20-60% by weight of the composition.

The wax component may comprise a mixture of two separate wax components, the first being a relatively low molecular weight material which provides moisturizing and the second being a higher molecular weight material which has a higher degree of adhesion to the skin than does the first component. The wax components may be esters of waxes. In particular instances, the wax component has a melting point in the range of 35-72° C., and may comprise, on a weight basis, 10-50% of the composition. In particular instances, the wax component comprises 15-30% of the composition. Jojoba derived waxes are utilized in specific embodiments, and jojoba esters are one material of this type which may be employed in particular formulations. The composition may also include water and may further include emulsifiers.

Also disclosed are methods for moisturizing and exfoliating the skin through the use of the compositions.

### DETAILED DESCRIPTION OF THE INVENTION

Embodiments of the present invention include an exfoliant and moisturizing compositions for use as personal care products, such as on the body, feet or face of the individual (also referred to as either subject or user). Embodiments can have the form of a stick, tablet, or other suitable solid or semisolid format. Compositions are described that contain waxes derived from various animal, fruit, plant, synthetic and vegetable sources; moisturizers, minerals and exfoliant particles. As will be described, the exfoliant particles are mainly particles having a fine size (generally less than 3 microns). This combination of minerals, clay, plant waxes and emulsifier acts as both moisturizer and exfoliant. Mineral exfoliants aid the exfoliation process. The user can also visually see the degree of exfoliation and dead skin (along with product) falling off the skin. In spite of effectiveness of the product, this combination does not promote dry skin, or irritation. This combination is mild and gentle to the skin.

The composition has a “self-regulating” property such that the skin is exfoliated to the extent needed by the skin itself. The inventive exfoliating composition visually shows the user that the exfoliation process is working and what area of the body needs further exfoliation. This is evidenced by the size and configuration of particles produced white using embodiments of the present invention. As the composition is being used, in conjunction with water and perhaps with a glove, cloth or mitten, rather than a lather being produced, as skin cells are being exfoliated, they are accumulated into particles of combining the exfoliant composition and the removed cells. The size and quantity of the particles is a visual indication of exfoliation.

Embodiments of the present invention also include methods of manufacturing the composition, in which:

a. Composition can be extruded;

b. Composition can be hot poured into solid product form; or

c. Composition can be pressed into a product form, such as a tablet.

The combination of wax and exfoliating particles both enhance the exfoliating process (desquamation). It is believed that higher molecular weight waxes in the composition attach themselves to the dead cells while the lower molecular weight waxes in the combination provide moisturization. The fine minerals provide the mechanical abrasion and the gentle cleansing process. The fine particle minerals in the composition do not damage the skin or irritate, as some commercially available exfoliants do. The combination of the waxes, moisturizers, and exfoliants provide the user with simultaneous moisturization and exfoliation.

Embodiments of the composition of the present invention include a naturally derived nonionic emulsifier, natural wax esters, emollients, anionic clays and minerals. Each of these components, as well as optional or additional components, is described in detail hereinafter.

All percentages, parts and ratios are based on the total weight of the composition, unless otherwise specified.

The term “solid particle” as used herein means a particle that is not a liquid or a gas.

### Anionic Particles

Embodiments of the composition of the present invention may include solid anionic particles dispersed throughout the composition to form a homogeneous product. Such solid anionic particles include hydrated aluminum silicates and carbonates, magnesium salt, and combinations thereof.

It has been found, therefore, that the solid anionic particles described herein can be formulated into the composition of the present invention to provide long lasting or enduring moisture to the skin. Agents such as hydrated aluminum silicate are known to help the moisture absorbing capacity of skin by adhering to the skin and acting as a “magnet” for moisture from the skin or other applied area after application.

### Waxes

The waxes and/or oils are selected from one or more animal, fruit, plant, synthetic or vegetable sources. Embodiments can include one or more waxes and one or more oils. An animal-derived wax, lanolin, can be used in the range of from about 5% to about 20% of the composition, and is used in the range of from about 10% to about 15% of the composition in certain embodiments. Sorbitan palmitate (SPAN® 80, ICI Americas, Wilmington Del.) could be used in the range of from about 1% to about 10% of the composition, and used in the range of from about 1% to about 5% of the composition in certain embodiments. A vegetable extract used is one of several forms of jojoba oil (from Simmondsia chinensis). In some instances, a jojoba wax is prepared by hydrogenation of jojoba oil, and this fully hydrogenated wax is characterized by having a melting point of from about 68° C. to about 72° C. An isomerized preparation of jojoba oil is also used; this preparation is referred to as an oxidative, stable jojoba butter, and is characterized by having a melting point (about 35° C.) that is considered to be close to that of normal human body temperature. Both products are commercially available (Desert Whale, Tucson, Ariz.). The isomerized jojoba oil, obtained as ISOJOJOBA™ 35, could be used in the range of from about 5% to about 20% of the composition, and is used in the range of from about 5% to about 12% of the composition. The hydrogenated jojoba oil, obtained as a jojoba wax, could be used in the range of from about 5% to about 20% of the composition, and is used in the range of from about 6% to about 12% of the composition.

In yet other instances, the wax component is an ester of jojoba, such as the material known in the art as jojoba ester-70. Jojoba esters are wax members of the lipid family. Natural jojoba is a mixture of long chained, unbranched liquid wax esters that result from the esterification of an omega-9, monounsaturated linear fatty acid and an omega-9 monounsaturated linear fatty alcohol. The dominant fatty alcohols and fatty acids of jojoba are of C20 and C22 in length. The unbranched, long chain nature of the alcohol and acid moieties give jojoba esters their nonpolar behavior. Natural jojoba, as well as fats and oils from plants, all contain tocopherols which are known to act as free radical scavengers. Jojoba ester-70 significantly increases skin softness, effectively moisturizes and softens the skin by a dual action of forming a lipid layer which is partially occlusive and by the diffusion of jojoba into the intercellular spaces of the stratum corneum to soften the tissue.

### Minerals

The minerals used are one or more of the following, such as a hydrated aluminum silicate, which may be in the form of either kaolin, bentonite, hectorite, montmorillonite, laponite, or as mica, talc or starch. GHASSOUL Clay (Alban Muller International, Vincennes, France) is one type of clay which is used in the range of from about 1.00% to about 15% of the composition, and is also used in the range of from about 1.00% to about 12% of the composition. A starch, such as a tapioca starch sold under the trademark of NATRASORB® (National Starch, Bridgewater N.J.), used in the range of from about 2% to about 30% of the composition, is also used in the range of from about 3% to about 13% of the composition.

The hydrated aluminum silicate can be used in the range of from about 15% to about 40% of the composition, and is used in the range of from about 18% to about 30% of the composition. Another mineral, such as a carbonate, such as calcium carbonate or zinc carbonate, could be used. In an embodiment of the present invention, calcium carbonate could be used in the range of from about 20% to about 40% of the composition, and is used in the range of from about 28% to about 35% of the composition. Stearic acid, in the form of a stearate salt, such as magnesium stearate or zinc stearate, is included to provide adhesion and slip to the skin, and could be used in the range of from about 1% to about 5% of the composition.

### Conventional Adjunct Ingredients

Embodiments of the composition of the present invention may further comprise other adjunct ingredients that may modify the physical, chemical cosmetic or aesthetic characteristics of the composition or serve as additional “active” components when deposited on the skin. Many such adjunct ingredients are known for use in personal care compositions, and may also be used in the topical compositions herein, provided that such adjunct materials are compatible with the essential materials described herein, or do not otherwise unduly impair product performance.

Such adjunct ingredients are most typically those materials approved for use in cosmetics and that are described in reference books such as the CTFA, Cosmetic Ingredient Handbook, The Cosmetic, Toiletries, and Fragrance Association, CTFA International Buyers Guide. Non-limiting examples of such adjunct ingredients include preservatives, chelating agents for metal ion control, antioxidants, fragrance, co-solvents, emollients, vitamins, and combinations thereof.

A chelating agent, such as tetrasodium ethylenediamine tetracetic acid (Na₄EDTA) is added to the composition in concentrations ranging from about 0.01% to about 0.25%, and generally at a concentration ranging from about 0.05% to about 0.20%. The chelating agent can be obtained from a variety of commercial sources; Akzo Nobel (Chicago, Ill.) provides a form of Na₄EDTA sold under the trademark DISSOLVINE® 240S. One or more preservatives are added to the composition, and may be chosen from among the preservatives conventionally used for cosmetics and skin care products. OPTIPHEN™ is a preservative comprising a combination of 2-phenoxyethanol and 1, 2 octanediol, does not include any paraben, and is used at a concentration ranging from about 0.5% to about 1.5% of the composition. In certain embodiments, OPTIPHEN™ is employed at a concentration of from about 0.5% to about 0.7% of the composition. Other preservatives which contain one or more parabens, such as methyl paraben, butyl paraben, ethyl paraben, or propyl paraben, either alone or in combination with another preservative, such as 2-phenoxyethanol, can also be utilized. Such compounds are sold under the trademarks of PHENONIP® (Clariant, United Kingdom) or GERMABEN® (International Specialty Products).

Antioxidants can also be added to the composition as a preservative, and can be obtained from a number of commercial sources. Tetradibutyl pentaerithrityl hydroxyhydrocinnamate, such as that sold under the trademark CIBA® TINOGARD® TT or CIBA® TINOGARD® TTDD (Ciba Specialty Chemicals, High Point, N.C.) is added to an embodiment of the composition in concentrations ranging from about 0.01% to about 0.30%, and generally in the range of from about 0.01% to about 0.07%.

A non-ionic glucoside, used in the form of a mixture of a glucoside and its alcohol, is added to the composition where this mixture serves as an emulsifier, and is also known to act as a texturizing agent. In one example, a mixture of myristyl alcohol and myristyl glucoside is used in a concentration ranging from about 0.5% to about 2.5% of the composition, and is generally used in a concentration ranging from about 0.65% to about 1.8% of the composition. In one embodiment, this glucoside mixture is obtained commercially, as a product sold under the trademark of MONTANOV® 14 (Seppic, Paris, France). Other glucosides, such as cetearyl glucoside, cocoyl glucoside or arachidyl glucoside can also be used, and aid in the skin feel and product performance of particular embodiments. Such glucoside mixtures are also obtained commercially, such as those products sold under the MONTANOV® trademark.

Another stabilizer/emulsifier which may be used in compositions of this type comprises an oil-in-water liquid crystal gel network material comprised of sorbitan stearate and sorbityl laurate. Such materials are commercially available under the name “Arlatone LC”. These liquid crystal gel networks have micellar features in the range of 20 to 50 microns and form a sponge-like structure which stabilizes the oil and aqueous phases of the composition. This structure traps moisture and slowly releases it to the skin, thus aiding in the exfoliation process of the clays.

The fine minerals provide the mechanical abrasion and gently cleansing process. The fine particle size of the minerals (0.60-3 microns) does not damage the skin as some commercially available exfoliants do (e.g. apricot seeds). Embodiments of the present invention, in either an exfoliating stick, tablet, solid, semisolid or other product form, leave the user's exfoliated skin appearing fresh and healthy as it removes the dull layer of dead skin, accompanied with non-soap cleansing, leading to less clogged pores while at the same time moisturizing the skin to minimize irritation and dryness.

Embodiments of the present invention include a method of manufacturing an exfoliating and moisturizing composition. To achieve exfoliation with gentle cleansing and simultaneous moisturizing, the combination of oil and water emulsion is blended together with the clays and minerals to form a homogeneous product. As will be described, the product is manufactured using an extrusion process. This process enables the inventive compositions to be produced in a novel form with a high concentration of fillers.

When embodiments of the present invention are used, one can obtain increased efficacy of exfoliation when they are used in conjunction with either a cloth, sponge, mitt or glove, one or more of which can be combined with an embodiment of the present invention and sold as a kit, or which can be marketed as separate items, and the individual can select which item they prefer to use. Thus, an individual can use an embodiment of the present invention while, for example only, washing their face or other body part near a sink, while bathing or showering, after a sauna, under conditions that are characteristic of those of “Turkish baths”, or in other ways as desired by a particular individual.

The following examples further describe and demonstrate the embodiments within the scope of the present invention. The examples are given solely for the purpose of illustration, and are not to be construed as limitations of the present invention since many variations thereof are possible without departing from its scope.

### Example 1

### Preparation of an Exfoliating Composition

Exemplary embodiments have compositions summarized in Table 1.

COMPOSITION EMBODIMENTS

| | Example 1 | Example 2 | Example 3 | Example 4 | Example 5 | Example 6 | Example 7 | Example 8 | Example 9 |
|---|---|---|---|---|---|---|---|---|---|
| Phase A | | | | | | | | | |
| Deionized Water | 18.00% | 20.00% | 21.18% | 20.45% | 20.32% | 20.31% | 10.00% | 20.31% | 20.34% |
| Tetrasodium EDTA a | 0.10% | | | | 0.11% | 0.11% | | 0.11% | |
| Phase B | | | | | | | | | |
| Sorbitan Palmitate b | | 3.00% | | | | | | | |
| Jojoba Oil, Isomerized c | 7.00% | | 5.88% | 10.23% | 7.90% | 7.90% | | 10.16% | 10.17% |
| Myristyl Alcohol and | 1.50% | | | | | | | | |
| Myristyl Glucoside d | | | | | | | | | |
| Jojoba Wax e | 10.00% | | 14.12% | 9.09% | 11.28% | 11.28% | | 9.03% | 9.04% |
| Lanolin Wax f | | 13.00% | | | | | 15.00% | | |
| 2-Phenoxyethanol g | 0.50% | | | | 0.56% | 0.56% | | 0.56% | 0.56% |
| Tetradibutyl Pentaerithrityl | | | | | 0.02% | 0.06% | | 0.02% | |
| Hydroxyhydrocinnamate h | | | | | | | | | |
| Phase C | | | | | | | | | |
| Calcium Carbonate FCC | 30.00% | 28.50% | 35.29% | 34.09% | 33.85% | 33.84% | 65.00% | 33.85% | 33.90% |
| Grade | | | | | | | | | |
| Kaolin, USP BC | 20.00% | 29.50% | 23.53% | 22.73% | 22.57% | 22.56% | | 22.57% | 22.60% |
| Magnesium Stearate NF | | 6.00% | | 3.41% | 3.39% | | | 3.39% | 3.39% |
| Bentonite Clay i | | | | | | | 10.00% | | |
| Tapioca Starch j | 12.90% | | | | | 3.38% | | | |
| | 100.00% | 100.00% | 100.00% | 100.00% | 100.00% | 100.00% | 100.00% | 100.00% | 100.00% |

a. DISSOLVINE ® 240S (Akzo-Nobel, Chicago IL)
b. SPAN 80 (ICI Americas, Wilmington, DE)
c. ISOJOBA ™ 35 (Desert Whale, Tucson AZ)
d. MONTANOV ® 14 (Seppic, Paris, France)
e. JOJOBA OIL, HYDROGENATED (Desert Whale)
f. LANOLIN WAX (Rita Corporation, Crystal Lake, IL)
g. PHENONIP ® (Nipa Hardwicke Inc., Wilmington, DE)
h. CIBA ® TINOGARD ® TT or CIBA ® TINOGARD ® TTDD (Ciba Specialty Chemicals, High Point, NC)
i. GHASSOUL (Alban Muller International, Vincennes, France)
j. NATRASORB ® (National Starch, Bridgewater, NJ)

### Example 2

### Method of Manufacture

The examples previously described are prepared as follows. The materials for Phase C are mixed until the phase is uniform. The materials for Phase A are prepared, mixed and heated at a temperature of about 75° C. The emollients, waxes and emulsifiers of Phase B are then added to Phase A with mixing. The resulting batch is then heated to about 80° C. with mixing until it becomes homogeneous. Preservative and antioxidants, as required for a specific embodiment, are added with continued mixing. The emulsion phase and Phase C are combined together with mixing, which is continued until the batch starts to cool and has a “dough-like” consistency. The resulting product is processed through an extruder containing a mesh screen. The mesh screen is a standard mesh screen, selected from screens having a size ranging from 10 mesh to 50 mesh. In certain embodiments, the resulting product may be reprocessed through the extruder multiple times in order to produce a composition having a solids content higher than can be obtained in a single extrusion step. Depending upon the final intended product configuration, the composition is processed further.

When the final product is to be used as a tablet, a quantity is pressed using a Carver Press under pressure, ranging from about 10,000 psi to about 25,000 psi, more specifically a pressure ranging from about 12,000 psi to about 25,000 psi of pressure. The size of a tablet can range from about 10 grams to about 25 grams, and generally the size ranges from about 12 grams to about 16 grams, although other size tablets can be utilized.

The extruded product can be cut and inserted into a stick container. When the extruded product is put into a stick container, the top surface of the stick can be given a smooth finish using conventional processes.

Where the final product is intended for use as a stick, it is poured into the container or mold, as appropriate, for a stick, at a temperature of from about 75° C. to about 80° C.

### Example 3

### Effects of Exfoliant Composition: Skin Irritability Test

To determine whether embodiments of the present invention could induce irritation and/or allergic contact sensitization through repetitive skin contact, testing was done on human subjects. This study employed a three week induction phase and a two week challenge phase. A skin patch was dosed with a sample of material sufficient to cover the test area, between the scapulae, of the subjects. During the induction phase, samples were applied three times weekly, with each site evaluated prior to reapplication of the test material. If a test site showed a reaction of ≧2, application was moved to another site. In a three day challenge phase at the end of the induction period, samples were applied to new test sites which were then evaluated after 24 hr and after 72 hr. The sites were graded on the order of 0 to 4, where 0=no skin response; 0.5 a barely perceptible erythema; 1 a mild, pink uniform erythema covering most of the patched area and/or edema; 2 a moderate pink red erythema, uniform in the patched area and/or edema; 3 a marked bright red erythema with or without petechiae or papules and/or edema, and 4 a severe deep red erythema with or without vesiculation or weeping and/or edema. The results of this study, using a composition including jojoba oil and jojoba wax, did not indicate a potential for dermal irritation or a potential for allergic contact sensitization.

The foregoing discussion and description illustrates some specific embodiments of the present invention. In view of the teaching herein, yet other embodiments, modifications and variations of the invention will be apparent to those of skill in the art. Therefore, the foregoing is understood to be illustrative of, but not limited upon, the principles of the present invention. It is the following claims, including all equivalents, which define the scope of the invention.

1. An exfoliating and moisturizing composition for the skin, said composition consisting of:
a wax component having a melting point of at least 35° C.;
an anionic exfoliate mineral component dispersed in said wax component, said mineral component having a particular size of no more than 3 microns; and
optionally at least one member selected from the group consisting of, starch, water and adjuncts.

2. The composition of claim 1, wherein said mineral component includes a clay.

3. The composition of claim 2, wherein said clay is selected from the group consisting of bentonite, kaolin, hectorite, montmorillonite, laponite, and combinations thereof.

4. The composition of claim 1, wherein said mineral component includes a salt of a metal selected from the group consisting of Al Mg, Ca, Zn, and combinations thereof.

5. The composition of claim 4, wherein said salt is selected from the group consisting of: silicates, carbonates, fatty acid salts, and combinations thereof.

6. The composition of claim 1, wherein said mineral component has a particle size in the range of 0.6-3.0 microns.

7. The composition of claim 1, wherein said mineral component comprises, on a weight basis, 3-80% of said composition.

8. The composition of claim 1, wherein said mineral component comprises, on a weight basis, 20-60% of said composition.

9. The composition of claim 1, wherein said wax component comprises a first wax component having a first molecular weight, said first component being a moisturizing component; and a second component having a second molecular weight which is greater than said first molecular weight, said second component having a higher degree of adhesion to the skin than does the first component.

10. The composition of claim 9, wherein at least one of said components is an ester of a wax.

11. The composition of claim 1, wherein said wax component has a melting point in the range of 35-72° C.

12. The composition of claim 1, wherein said wax component comprises, on a weight basis, 10-50% of said composition.

13. The composition of claim 1, wherein said wax component comprises, on a weight basis, 15-30% of said composition.

14. The composition of claim 1, wherein said wax component is derived from jojoba.

15. The composition of claim 1, further including 5-30% by weight of water.

16. The composition of claim 1, further including starch.

17. Method for moisturizing and exfoliating the skin, said method comprising the steps of:
providing a compensation consisting of a wax component having a melting point of at least 35° C.; an anionic exfoliating mineral component dispersed in said wax component having a particulate size of no more than 3 microns; and optionally, at least one member selected from the group consisting of starch, water and adjuncts;
wetting the skin;
applying said composition to the skin; and rubbing said composition into the skin whereby said composition moisturizes the skin and exfoliates dead skin cells.

18. The method of claim 17, wherein said exfoliating composition comprises by weight 10-50% of a wax component having a melting point in the range of 35-72′; 3-80% by weight of a mineral component having a particle size in the range of 0.6-3.0 microns.

19. The method of claim 17, wherein said mineral component comprises a clay.

20. An exfoliating and moisturizing composition for the skin, said composition comprising:
a wax component having a melting point of at least 35° C.; and
an exfoliant mineral component dispersed in said wax component, said mineral component having a particle size of no more than 3 microns.
